(19) United States
(12) Patent Application Publication
(10) Pub. No.: US 2025/0375109 A1
(43) Pub. Date: Dec. 11, 2025

(54) METHOD FOR CHECKING A MAGNETIC RESONANCE LIMIT VALUE BASED ON A POSITION OF A HEAD OF A PATIENT

(71) Applicant: Siemens Healthineers AG, Forchheim (DE)

(72) Inventors: Thomas BECK, Dormitz (DE); Sven HELMECKE, Nuernberg (DE)

(73) Assignee: Siemens Healthineers AG, Forchheim (DE)

(21) Appl. No.: 19/227,588

(22) Filed: Jun. 4, 2025

(30) Foreign Application Priority Data
Jun. 5, 2024 (DE) 10 2024 205 164.0

(51) Int. Cl. A61B 5/00; A61B 6/04; A61B 8/00

(52) U.S. Cl. CPC A61B 5/0033; A61B 6/0407; A61B 8/4254

(57) ABSTRACT

One or more example embodiments relates to a method for position determination during a magnetic resonance examination and checking a medical limit value. The method comprises determining position information of a specific part of a body of a patient on a patient table; and checking the magnetic resonance sequence for adherence to the at least one limit value using the position information of the specific part of the body. One or more example embodiments makes possible a specific determination of the absorption rate of specific parts of the body and an increase in the performance of a magnetic resonance apparatus.

### CROSS-REFERENCE TO RELATED APPLICATION(S)

The present application claims priority under 35 U.S.C. $119 to German Patent Application No. 10 2024 205 164.0, filed Jun. 5, 2024, the entire contents of which are incorporated herein by reference.

### FIELD

One or more example embodiments relates to a method for position determination during a magnetic resonance examination and for checking a medical limit value.

### RELATED ART

During a magnetic resonance examination of a patient, i.e. when carrying out Magnetic Resonance Imaging, (MRI), magnetic fields, in particular gradient fields, and radio-frequency signals or radio-frequency fields in accordance with a measuring protocol are usually employed for acquisition of magnetic resonance signals by a magnetic resonance apparatus. For generation of the gradient fields a magnetic resonance apparatus usually has a gradient coil unit. The magnetic resonance apparatus further usually comprises a radio-frequency antenna unit, with which the radio-frequency signals for exciting atomic nuclei can be generated.

Within the framework of an MR measurement it is of central importance to ensure the safety of the patient during the overall course of the examination. The safety measures are usually oriented to standards, such as IEC 60601-1 and also IEC 60601-2-33. Within the framework of these standards inter alia physical parameters are determined, which are to be complied with to ensure the safety of the patient. In particular, a specific absorption rate (SAR) is often of decisive relevance as a parameter. The SAR value or parameter usually characterizes the amount of energy that is absorbed by an object during a magnetic resonance examination. A high SAR value leads to a strong heating up of the tissue. Therefore, it is of importance which part of the body is localized during the examination period in the isocenter, at the point of the most concentrated radiation. Different limit values or highest values have been determined empirically for different regions of the body of patients. Typically the head of a patient, with 3.2 Watts per kilogram is the part of the body with the highest absorption rate. The positioning of the head of a patient is accordingly a deciding factor during a magnetic resonance examination and must be able to be determined as precisely as possible.

Typically, in magnetic resonance examinations there must be a decision taken between two support options of the patient: head-first or feet-first. When the patient is supported in the head-first position the position of the head is primarily uniquely defined by the use of a head coil. The head coil is located in this case by a mechanical connection with the patient couch at a position always defined. This is not the case with a feet-first position of a patient, since with this support variant a head coil is not usually used. This means that no mechanical boundary conditions for a unique positioning of the head are present. In order now to ensure that the SAR values are safely adhered to, in particular for the head area, there is an estimation of the position of the head during the examination. In order to avoid the absorption values being exceeded in any case it is assumed that the head of the patient is arranged in each patient couch position in the isocenter of the magnetic resonance apparatus.

### SUMMARY

This procedure merely represents a rough estimation of the position and therefore leads to performance issues during the examination.

One or more example embodiments increases the performance of the magnetic resonance apparatus during the examination of a patient while simultaneously adhering to defined safety measures for the patient. The is achieved by the features of the independent claims. Advantageous embodiments are described in the dependent claims.

### BRIEF DESCRIPTION OF THE DRAWINGS

Further advantages, features and details emerge from the exemplary embodiments described below, as well as with the aid of the drawings. Parts that correspond to one another are labeled with the same reference numbers in all figures. A repetition of the description of parts that correspond to one another in the respective exemplary embodiments is dispensed with. Exemplary embodiments can essentially be distinguished from one another by the arrangement of the units.

In the figures:

FIG. 1 shows a head-first position of a patient in a magnetic resonance apparatus with a position determination apparatus in accordance with one or more example embodiments,

FIG. 2 shows a process flow of a check of a magnetic resonance sequence for adherence to at least one limit value in accordance with one or more example embodiments,

FIG. 3 shows a feet-first position of a patient in a magnetic resonance apparatus with a position determination apparatus and a foreign object in accordance with one or more example embodiments,

FIG. 4 shows a feet-first position of a patient in a magnetic resonance apparatus with a position determination apparatus in accordance with one or more example embodiments,

FIG. 5 shows a process flow of a check on a magnetic resonance sequence for adherence to at least one limit value with further method steps in accordance with one or more example embodiments, and

FIG. 6 shows a process flow of a check on a magnetic resonance sequence for adherence to at least one limit value with intermediate steps in accordance with one or more example embodiments.

### DETAILED DESCRIPTION

The proposed way in which the object is achieved both with regard to the claimed apparatuses and also with regard to the claimed method will be described below. Features, advantages or alternate forms of embodiment are also likewise to be transferred to the other claimed subject matter and vice versa. In other words, the physical claims (which are directed to an apparatus for example) can also be developed with features that are described or claimed in conjunction with a method. The corresponding functional features of the method are embodied by corresponding physical modules.

A method for checking a magnetic resonance sequence for adherence to at least one limit value is proposed. The method comprises a determination of position information of a specific part of the body of a patient on a patient table. Moreover, the method comprises checking of the magnetic resonance sequence for adherence to the at least one limit value with the aid of the position information of the specific part of the body.

The specific part of the body of the patient can in particular be the head of the patient. The specific part of the body can however also comprise other parts of the body of the patient, for example the feet of the patient, the chest area, the torso and/or the heart (the area surrounding the heart) of a patient. Example embodiments of the invention are described below especially with the aid of the head of the patient as the specific part of the body. Likewise, the claimed aspects are to be transferred and/or able to be applied to the further specific parts of the body of a patient.

A magnetic resonance sequence typically designates a pulse sequence, i.e. a chronological sequence of the radio-frequency pulses and/or gradient pulses for exciting an image volume to be measured, for signal generation and spatial encoding. Typical pulse sequences in this case can comprise spin echoes, in particular turbo spin echoes (TSE), and/or gradient echoes.

The (checked) limit value preferably comprises a SAR (“specific absorption rate”) value, in particular a body part SAR value, and/or a SED (specific energy dose) value. The limit value can also comprise other medical values. The SAR value usually represents a safety value or parameter during a magnetic resonance examination. The specific absorption rate in this case is usually the value of absorbed radio-frequency energy per unit of time and per kilogram of body weight. The absorption of the radio-frequency energy can lead to heating up of the body tissue. The energy absorption is preferably an important variable for the definition of the safety limit values. With an impermissibly high concentration of radio-frequency energy radio-frequency burns can occur, which is why preferably the determination of local SAR values, such as for example at the head of a patient, takes place. With even distribution of the radio-frequency energy over the entire body the load on the thermo-regulation or the heart circulation system of the patient is considerable. Therefore there is also preferably the determination of the whole body SAR. Possible remedial measures for SAR values that are determined as too high can comprise: Use of radio-frequency pulses that result in a lower SAR value, smaller flip angles, higher repetition times (TR), and/or fewer imaging slices. The specific energy dose (SED) represents a further possible safety value or parameter during a magnetic resonance examination. The specific energy dose in this case is typically the value of the accumulated whole-body SAR during the entire examination, often specified in J/kg (=Ws/kg). The body part SAR value is a further possible safety value or parameter during a magnetic resonance examination. The part body SAR values typically refer to the SAR value which is averaged per unit of time over the body mass of a patient exposed to a volume coil (radio-frequency transmit coil).

Preferably the position information can describe the (ideally actual) spatial location of the head of the patient on the patient couch. Preferably the position information can comprise a three-dimensional and/or two-dimensional description (in x and y) of a point, in particular of a midpoint, of the head, starting from a reference object, in particular a reference point, by means of a coordinate system. The reference point (for example null point of a coordinate system) in this case is preferably arranged on or at the patient table. The position of the head can preferably be determined by means of the midpoint of the head, but also by means of another point located at/on the head of the patient, for example the vertex (in particular of the point at which the local maximum of the function lies, which describes the upper half of the skull of the patient in the state viewed from the front).

Magnetic resonance apparatuses preferably have a patient receiving area. During the examination of the patient the patient is typically located entirely or partly in the patient receiving area. Preferably the patient area is of a cylindrical design and/or embodied in a tubular shape. Preferably the patient area is moreover surrounded by a gradient coil unit and a main magnet. Preferably a patient support apparatus, in particular a patient table, can be moved into the patient receiving area. The patient table can preferably accommodate a patient lying horizontally. The patient table can comprise electronic components and connections, which can for example make possible a change in the position (or a movement) of the patient table. The patient receiving area can also be referred to as the examination area and/or comprise the examination area. The examination area here is in particular the area in which an imaging examination of the patient takes place.

The determination of the position information of the head of the patient on the patient table can comprise a measurement, acquisition and/or determination of the position of the head of the patient. In this case the position information is preferably measured, acquired and/or determined by means of a sensor embodied for this purpose. In particular the determination of the position can be undertaken with the aid of measurement data, which can be acquired by means of the sensor embodied for this purpose.

The checking of the magnetic resonance sequence for adherence of the at least one limit value is undertaken with the aid of the position information determined, in particular position data, of the head of the patient. Preferably the checking comprises a determination and/or calculation of an absorption value, in particular of a SAR value, with the aid of the position information. Here for example a Lookup Table (LUT or conversion table), which assigns one or more absorption values to the position information, in particular the position data, of the head, in particular taking into consideration method parameters, is used. Preferably the checking moreover comprises a comparison of a limit value, in particular of a SAR limit value, with a determined absorption value.

Determining the position information of the head of the patient advantageously enables the corresponding local loading and/or stress on the area of the body to be determined and enables it to be ensured that said area is not exceeding a specified limit value. An estimation of the head position can hereby be replaced and thus the performance of the system enhanced, since possibly higher loads or stress on areas of the body well away from the head become possible.

In accordance with one or more example embodiments, the method comprises a determination of distance measurement data for measuring a distance between a target object and a reference object, in particular a reference point, of the patient table. The target object in particular involves the head of the patient. In accordance with the aspect, the determination of the position information of the target object, in particular of the head, is advantageously undertaken in this case with the aid of the distance measurement data.

The distance measurement data in this case comprises at least data of a measurement of a distance between the target object (measurement object) and a reference point. Preferably the data of a number of measurements can also be acquired. The target object can in particular be the head of the patient. The presence of other objects on the patient table, such as for example covers, pillows, paper towels or ear defenders (headsets), means that instead of the distance to the head of the patient starting from the reference point, the distance to other objects can be the measurement result. This is in particular the case when another object, which for example is needed or used during the examination, is placed between the head of the patient and the reference point on the patient table. For example, the patient or medical personnel can put an object down on the patient table during the examination.

The reference object, in particular the reference point, is preferably the starting point for the distance measurement, i.e. for the acquisition of the distance measurement data. The reference object, in particular the reference point, can preferably be stationary or at a fixed location on the patient table. The reference object, in particular the reference point, can alternatively also be movable or drivable (in defined relationship) with the patient table. The reference object is in particular a point arranged on the patient table. As an alternative, the reference object can also comprise a line arranged on the patient table or an area of the patient table. In particular the spatial location of reference object is known, for example in a (global) coordinate system of the magnetic resonance apparatus, or can be determined. The determination of the position information can preferably comprise a grouping, in particular an addition, of the position information of the reference point and of the distance measurement data. In particular the reference object can be the null point of a patient coordinate system or be arranged at a point of the patient coordinate system. A patient coordinate system is preferably a patient-related coordinate system, which is embodied to show the location of a patient (or a slice in MR images) in the direction of view of an observer. Preferably the axes of the patient coordinate system run sagittally from right to left, transversally from head to foot and coronally from anterior (bottom) to posterior (top).

Distance measurements typically represent a robust, precise and reliable measuring method, so that the position of the head can advantageously be determined with adequate accuracy and safety.

In accordance with one or more example embodiments, the acquisition of the distance measurement data is undertaken by means of a distance sensor.

In other words, the distance data is preferably measured by means of at least one distance sensor. As an alternative a number and/or different distance sensors can also be used. Distance sensors can, inter alia, also be referred to as displacement measurement sensors, displacement sensors, position sensors, position probes and/or distance sensors. The distance sensor is preferably embodied to determine a spacing (distance) to a measurement object, starting from a reference object, in particular reference point (measurement starting point), or between two points (or objects). The reference object or the reference point in this case preferably corresponds to the position of the sensor. The distance sensor can preferably measure and/or determine the spatial position of a measurement object. The distance sensor can have a defined measurement range and/or a specific measurement accuracy. The distance sensor can preferably be embodied to detect the change in position of a (target) object and/or to acquire distance measurement data dependent on time. The distance sensor can in particular be a non-contact distance sensor. The distance sensor can preferably be an optical distance sensor. The distance sensor can for example be a laser distance sensor, a laser time-of-flight sensor, a confocal chromatic sensor, an interferometer, a capacitive distance sensor or an eddy current sensor. Tactile or contact sensors, such as for example touch probes, can represent an alternative, but lead to discomfort of the patient through their contact measurement and are therefore less preferable.

In particular non-contact distance sensors are therefore advantageously suitable for a distance measurement in the field of medical applications. The acquisition of the distance measurement data by means of a distance sensor is advantageously low-cost and robust.

In accordance with one or more example embodiments, the distance sensor comprises an ultrasound sensor.

In particular the distance sensor can be an ultrasound sensor. Preferably the ultrasound sensor comprises a transmitter, which is embodied to generate a sound wave, and a receiver, which is embodied to receive a sound wave. Preferably the distance of an object is calculated with the aid of the time in which the sound wave emitted by the transmitter and reflected by the measurement object is received by the receiver. The ultrasound sensor can in particular have a working range of between 20 centimeters and 5 meters. Since the attenuation of the sound can be dependent on ambient parameters, such as the air temperature, air humidity, air pressure, the parameters can be determined and taken into account in the course of the distance measurement. In particular the ultrasound sensor can be embodied to acquire spatially resolved information.

Advantageously ultrasound sensors make possible surface-dependent contactless distance measurements of different types of objects, in particular of the target object. Dust or other possible slight contaminations advantageously have no influence on the measurement. Moreover ultrasound sensors can also be of advantage in restricted or confined installation situations.

In accordance with one or more example embodiments, the distance measurement data comprises two and/or three-dimensional distance measurement data.

The two and/or three-dimensional distance measurement data can in particular represent an arrangement of measurement points within a measurement surface and/or a measurement space. Preferably two-dimensional distance measurement data comprises pairs of data assigned to each other consisting of an X and a Y value. Three-dimensional distance measurement data preferably moreover comprises a height value Z assigned to the XY data pair. In particular the distance measurement data can include the results of a number of distance measurements, starting from a reference object, in particular reference point. In other words, distance measurement data can be a listing (concatenation) of distances or distance vectors starting from (linked to) a reference point. The listing of the distance measurement data can preferably be stored in a document. The two and/or three-dimensional distance measurement data can preferably be displayed graphically to a user by means of a display unit. The two and/or three-dimensional distance measurement data can resolve a shape characteristic of an object, in particular of a head.

Through the acquisition of spatially-resolved distance measurement data, advantageously a more accurate position determination of the head (as the target object) and/or the recognition/differentiation (classification) of the head and other objects can be improved.

In accordance with one or more example embodiments, the method comprises an acquisition of object measurement data. According to the aspect there can preferably be a verification of the target object as the head of the patient with the aid of the object measurement data.

The object measurement data in this case in particular comprises the data of a measurement of a physical characteristic of an object, in particular of the target object. In other words, the object measurement data can in particular be the measured physical characteristics of an object, in particular of the target object. Preferably the object measurement data can comprise the temperature of an object, in particular of the target object. In particular the object measurement data can comprise the surface temperature of an object, in particular of the target object. Alternatively/in addition object measurement data can comprise the shape of an object, in particular of the target object, in particular the arrangement and shape properties of surfaces of an object, in particular of the target object. Alternatively/in addition object measurement data can comprise the surface properties, in particular a degree of absorption and/or degree of refection, and/or the surface roughness of an object, in particular of the target object. Alternatively/in addition object measurement data can comprise density properties of an object, in particular of the target object. Alternatively/in addition object measurement data, in particular of the target object, can comprise: Temperature characteristics of the object, for example a temperature curve, color properties of the object, in particular a color of a/the surface of the object.

With the aid of the object measurement data there can be an identification or verification of an object, in particular of the target object. The verification here is in particular a check by means of an objective means as to whether specific object characteristics are fulfilled. Here the identification or verification can preferably comprise a reconciliation (comparison) of expected values and the object measurement data. The expected values can be the physical characteristics of an object typically present (with spatial conditions), in particular of the target object. For example, the body temperature of a (healthy) person to be expected typically lies (under normal measurement conditions) at between 36.5 and 37.5 degrees Celsius (° C.). The head of a patient is thus preferably able to be distinguished from another object (located on the patient table), which for example has a room temperature of 23° C., by the determination of a temperature difference. The measurement conditions (ambient temperature, ambient pressure, air humidity) can preferably be taken into consideration in the measurement of the object measurement data. In particular the measurement conditions can be linked to the object measurement data or stored in a processing unit. In particular a check step can be provided, which for strongly deviating measurement conditions, checks the automatic detection or verification of an object, in particular of the target object.

If an image sensor is used as a verification sensor in order to generate image data of the object, in particular of the target object, the verification of the object, in particular of the target object, is preferably undertaken by an analysis of the image by application of transformation steps, for example of Fournier or Hough transformations. With the aid of the transformed image data an identification of the object, in particular of the target object, can be undertaken. In particular physical properties of the object, in particular of the target object, can only be recognized and/or verified with the aid of transformed image data. For example, by transformed image data of an image of a head of a patient, the hairs of the patient can be identified and thereby the object can be verified as the head of the patient. The object measurement data can make possible a unique identification and/or verification of an object, in particular as the head of the patient (as the target object). Physical properties of an object, in particular of the target object, can typically be determined in a safe and low-cost manner by means of established measurement methods. This can advantageously make possible a safe check or determination of the energy radiated into the head area of a patient. Thus, the safety of the examination is increased for a patient.

In accordance with one or more example embodiments, the acquisition of the object measurement data is undertaken by means of a verification sensor.

In other words, the measurement of the object data is preferably carried out by means of at least one verification sensor. As an alternative, a number of and/or different verification sensors can also be used. Verification sensors in the sense of example embodiments can also be referred to inter alia as identification sensors, object sensors or temperature sensors. The verification sensor can have a defined measuring range and/or a specific measuring accuracy. The verification sensor can be embodied to detect a characteristic change, for example a change in temperature, of an object, in particular of the target object or to detect object measurement data as a function of time. The verification sensor can preferably be a non-contact sensor. The verification sensor can preferably be an optical sensor. The verification sensor can preferably be a laser or light-based sensor. The verification sensor can for example be a camera, a temperature sensor or ultrasound sensor. For example, the verification sensor can be a depth image camera recording one-dimensional, two-dimensional and/or three-dimensional image data.

Verification sensors can advantageously make possible an (automatic) recognition and/or verification of an object, in particular of the target object, without involving (operating) personnel. In particular non-contact verification sensors are advantageously suitable for verification or identification of an object, in particular of the target object, in a medical examination context. The acquisition of the object measurement data by means of a verification sensor is advantageously low-cost, fast and reliable.

In accordance with one or more example embodiments, the verification sensor comprises a temperature sensor.

In particular the verification sensor can be a temperature sensor. The temperature sensor can also be referred to as a heat probe, temperature probe, heat sensor. In particular the temperature sensor can determine the temperature of an object, in particular of the target object, in a non-contact manner. The temperature sensor can in particular be embodied to be able to measure thermal radiation. For example, the temperature sensor can be a radiation thermometer, a pyrometer, an infrared sensor or a thermal image camera. Since the temperature measurements can be dependent on ambient parameters, such as the air temperature, air humidity, air pressure, these parameters can be determined and/or taken into consideration in the course of temperature measurement. In particular the temperature sensor can be embodied to detect spatially-resolved information.

Temperature sensors advantageously make possible a shape-dependent, fast (order of ms) and simple determination of an object temperature. In the medical context in particular non-contact temperature sensors due to freedom from feedback have (no mechanical) effect on the measurement object/the patient) and no hygiene requirements (no contact with the patient) in particular.

In accordance with one or more example embodiments, the object measurement data comprises two and/or three-dimensional object measurement data.

The two and/or three-dimensional object measurement data can in particular represent an arrangement of measurement points linked to physical characteristics with a measurement surface and/or a measurement space. Preferably two-dimensional object measurement data consists of a localization value (X/Y value) and a measurement value, for example a temperature value. Three-dimensional object measurement data preferably also comprises a height value Z assigned to the localization value. In particular the localization values are able to be determined starting from a reference value. In particular, the localization values comprise distance measurement data starting from a reference object, in particular reference point. The two- and/or three-dimensional object measurement data can preferably be displayed graphically to an operator by means of a display unit. The two and/or three-dimensional object measurement data can spatially resolve a shape characteristic of an object, in particular of a head (as the target object).

The acquisition of spatially-resolved object measurement data advantageously enables a more accurate object verification and/or the recognition/differentiation (classification) of objects, in particular of the target object, is made possible. Moreover, spatially-resolved object measurement data advantageously comprises additional distance information or distance measurement data.

In accordance with one or more example embodiments, the position information of the head (target object) is further determined with the aid of the object measurement data.

Preferably, the object measurement data can be employed not only for identification and/or verification of the object, but information from the object measurement data can be used for the determination of the position of the head. In particular, this can be undertaken with the use of spatially-resolving verification sensors. Distance information can preferably be determined from the object measurement data and compared and/or combined with the distance measurement data determined by a distance sensor. Similarly, the distance measurement data can preferably also be used for the recognition and/or verification of an object.

Advantageously, by the use of (the location information) of the object measurement data, the initial data situation for position determination can be expanded or improved. Advantageously the position of the head of the patient can be determined with improved safety and/or accuracy.

In accordance with one or more example embodiments, the method comprises provision of patient data. The determination of the position information of the head of the patient is preferably further undertaken in accordance with the aspect with the aid of the patient data.

For example, the patient data can be present in the form of an electronic (patient) record. Patient data can comprise anatomical information and/or measurement values, but also prior diagnoses or image data. For example, the patient data can comprise the body size, the head diameter, the body weight or the body temperature. By way of example, a check of the specific head position on the patient table can be carried out on the basis of the body size of the patient. With the aid of the head diameter, for example, the recognition of the head can be simplified and/or verified. Preferably the electronic patient data can be acquired from a processing unit, and the data relevant for the position determination provided.

Advantageously, the available patient information is noted and used for the position determination. Patient information can simplify both the position measurement and/or position determination and also the object measurement and/or object determination.

In accordance with one or more example embodiments, a defined estimated value can be defined as the position information. In accordance with the aspect, this can preferably be undertaken when the position information of the head cannot be determined with adequate safety and/or accuracy with the aid of measurement data.

Due to circumstances during the examination, for example through the positioning of a (non-detectable) object on the patient table, an incorrect position determination of the head can result or a (general) position determination cannot be possible. Despite this, in order to carry out an examination with errors that cannot be determined, there can be recourse to an estimated value as position information. The estimated value here is preferably determined so that the safety of the patient during the examination is guaranteed. This means that a “worst-case” scenario regarding the head position is used as a basis. The arrangement of the head here is in the isocenter, assumed to be the location of the magnetic resonance apparatus with the greatest local energy density acting on the patient.

Adequate safety and/or accuracy regarding the determination of the position information of the head with the aid of the measurement data refers in particular to reaching a defined safety value. The method can in particular comprise checking the (determined) position information of the head. The check of the position information can in particular produce a safety value, which can preferably be compared with the defined threshold value. The check on the position information of the head can in particular be undertaken by a trained function. The input data of the trained function in this case can in particular comprise the measurement data and the determined position information of the head. The trained function can preferably output a classification of the position information of the head into “safe” or “not safe”. As an alternative a check on the determined position value of the head can also be made by a user.

Preferably the estimation of the position information merely represents an option, which in the event of a fault advantageously makes at least an (inefficient) examination possible. In particular in situations in which a fast or timely examination of a patient is needed, an estimation can create considerable time benefits.

In accordance with one or more example embodiments, the determination of the position information of the head of the patient can preferably comprise a computer-implemented object classification method. The object classification method can comprise a trained function. The trained function can be embodied in this case to undertake a classification of the objects that could be recognized, determined and/or identified by means of the object measurement data. The classification of the objects can in particular take place into the following categories: Head of the patient, other object. Moreover, in accordance with the aspect an input of object measurement data into the trained function can be included. Moreover, in accordance with the aspect, a return to a classification of an object recognized by the trained function can take place.

The input data of the trained function can thus comprise two- and/or three-dimensional object measurement data and/or distance data. In particular, the input data can be present in the form of a file containing a point cloud. The input of the object measurement data can comprise a manual entry into a user interface or preferably an automatic entry or acceptance of the object measurement data through a first (input) interface. Preferably, a sensor detecting object measurement data, in particular a verification sensor, can convey object measurement data directly to the interface or be linked to said interface. The output of the classification information can in particular be undertaken via a second user interface or preferably via an (output) interface. Preferably, the output interface can be linked and/or connected to the user interface of the magnetic resonance apparatus embodied for carrying out the examination. The classification of the objects can preferably comprise further categories. In particular, the classification of the objects that cannot be assigned to the classification “head of a patient”, can take place in greater detail. For example, the further categories may comprise: Pillow, head support, other examination objects, other parts of the body (arms for example).

In other words, the method for checking a magnetic resonance sequence for adherence to at least one limit value can comprise a machine learning model. Machine learning models can also be referred to as a trained function, trained machine learning model, function with trained parameters, algorithm based on artificial intelligence or machine learning algorithm. In general, trained functions are embodied to generate defined output data by means of the processing of input data. In this case machine learning models emulate the cognitive learning processes that are typically associated with humans or with human brains. In particular, functions trained with training data can be employed in pattern recognition. The parameters of a trained function can in such cases in particular be updated at regular intervals and/or iteratively by training. Training methods usually used here comprise supervised training, semi-supervised training, unsupervised training or reinforcement training. In particular, the trained function can be trained by means of supervised and/or semi-supervised training. In particular, the trained function can comprise a neural network, a Support Vector Machine or a decision tree. In particular, the neural network here can be a deep neural network, a convolutional neural network or a convolutional deep neural network.

Advantageously, the checking as to whether the sensor data information acquired depicts a patient's head, in particular when spatially and/or temporally-resolved temperature and distance information are present, can be decisively improved by machine learning methods such as for example neural networks.

In accordance with one or more example embodiments, the determination of the head position information can comprise the provision of a trained function. The provision of a trained function can comprise the training of a function, for example of a neural network. The training of a function can in particular comprise: the input of training input data, preferably by means of a (first) training user interface, the input of (expected) training result data assigned to the training input data, preferably by means of a (second) training user interface, the training of the function by means of the training input data and training result data, preferably by means of a training computation unit, the provision of the trained function, preferably by means of a (third) training user interface. Advantageously, object identification and/or object verification can be improved by the training of the function.

Moreover, in accordance with one or more example embodiments, a magnetic resonance apparatus comprising a magnet unit, a patient table and a position determination apparatus is proposed. In accordance with the aspect, the magnet unit surrounds a patient receiving area. In accordance with the aspect, the patient table can be moved into the patient receiving area. In accordance with the aspect, the position determination apparatus can be embodied to carry out all steps or aspects of the described method for head positioning.

The position determination apparatus can in particular comprise a distance sensor and/or a verification sensor. The position determination apparatus can, for example however also only comprise one verification sensor or more than two sensors. The position determination apparatus can preferably be a component or module, consisting of the components distance sensor, verification sensor and a processing unit. Moreover, the position determination apparatus can preferably comprise a housing. In particular, the position determination apparatus can be connected to the system control unit and/or the user interface of the magnetic resonance apparatus.

A (conventional) magnetic resonance apparatus is advantageously able to be expanded with a position determination apparatus. Preferably an upgrade of an existing magnetic resonance apparatus is also possible. For example, the patient support apparatus can be replaced by a patient support apparatus comprising a position determination apparatus. As a consequence preferably there are no wide-ranging structural changes to the magnetic resonance apparatus and/or the patient support apparatus needed.

In accordance with a possible form of embodiment of the magnetic resonance apparatus, the position determination apparatus can be arranged on the patient table at one end of the patient table. In particular, the position determination apparatus can be arranged at a foot end of the patient table.

The patient table end in this case can in particular be the head end or foot end of the patient table. The end of the patient table, which, in the initial of the patient table is at a shorter distance from the magnet unit of the magnetic resonance apparatus, can in particular be referred to as the head end. The end of the patient table that is at a greater distance from the magnet unit in the initial location of the patient table can thus be referred to as the foot end of the patient table. Since the typically more frequently used, preferred patient support is the head-first position, it follows that preferably the positioning of the sensor system is at the foot end of the patient table. For a feet-first examination the patient is supported inverted on the patient table, meaning with their head at the foot end of the patient table.

Advantageously, the sensor is located at a position (at the end of the patient table), which is not moved into the patient receiving area (examination area). Through the positioning of the sensor system employed at a large spatial distance to the magnetic resonance system at the foot end of the patient table, a low-cost implementation and a simplified choice of the sensor system to be employed is made possible.

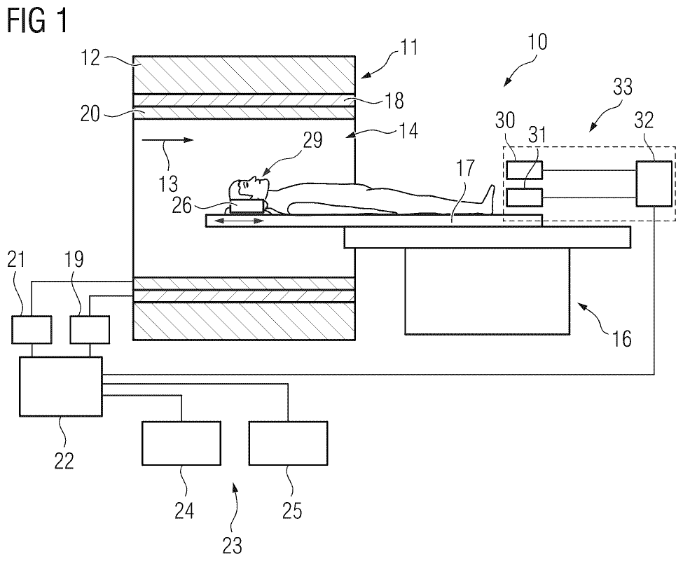

Shown schematically in FIG. 1 is a magnetic resonance apparatus 10. The magnetic resonance apparatus 10 comprises a magnet unit 11, which has a main magnet 12 for creation of a strong and in particular temporally constant main magnetic field 13. Moreover, the magnetic resonance apparatus 10 comprises a patient receiving area 14 for receiving a patient 15. The patient receiving area 14 in the present exemplary embodiment is embodied cylindrical in shape and surrounded in a circumferential direction by the magnet unit 11. Basically however an embodiment of the patient receiving area 14 that differs from this is always conceivable. The patient 15 can be pushed by means of a patient support apparatus 16 of the magnetic resonance apparatus 10 into the patient receiving area 14. To this end the patient support apparatus 16 has a patient table 17 able to be moved within the patient receiving area 14.

The magnet unit 11 furthermore has a gradient coil unit 18 for creation of gradient fields, which are used for spatial encoding during imaging. The gradient coil unit 18 is controlled by means of a gradient control unit 19 of the magnetic resonance apparatus 10. The magnet unit 11 furthermore comprises a radio-frequency antenna unit 20, which is embodied in the present exemplary embodiment as a body coil integrated permanently into the magnetic resonance apparatus 10. The radio-frequency antenna unit 20 is controlled by a radio-frequency antenna control unit 21 of the magnetic resonance apparatus 10 and emits radio-frequency magnetic resonance sequences into an examination space, which is essentially formed by a patient receiving area 14 of the magnetic resonance apparatus 10. Through this an excitation of atomic nuclei materializes for the main magnetic field 13 generated by the main magnet 12. Magnetic resonance signals are created by relaxation of the excited atomic nuclei. The radio-frequency antenna unit 20 is embodied for receiving the magnetic resonance signals.

For control of the main magnet 12, the gradient coil unit 19 and the radio-frequency antenna control unit 21, the magnetic resonance apparatus 10 has a system control unit 22. The system control unit 22 controls the magnetic resonance apparatus 10, such as for example the carrying out of a predetermined imaging gradient echo sequence. The system control unit 22 can moreover connect the radio-frequency antenna control unit 21 and the gradient control unit 19 and forward control commands to the corresponding control unit. Moreover, the system control unit 22 contains an evaluation unit, not shown in any greater detail, for evaluation of the magnetic resonance signals that are acquired during the magnetic resonance examination. Furthermore, the magnetic resonance apparatus 10 comprises a user interface 23, which is connected to the system control unit 22. Control information, such as for example imaging parameters and also reconstructed magnetic resonance images, can be displayed for a member of the medical operating personnel on a display unit 24, for example on at least one monitor of the user interface 23. Furthermore, the user interface 23 has an input unit 25, by means of which information and/or parameters can be entered by the medical operating personnel during a measurement process.

The patient 15 is located in FIG. 1 in a position referred to as the head-first position on the patient table 17. The support of the head of the patient 29 is undertaken by a head coil 26. The head coil 26 is mechanically attached at a (spatially) defined position, for example screwed, gripped or clamped. The position of the head of the patient 29 is thus, even with a movement of the patient table 17 by the patient support apparatus 16, always defined or known by the head coil 26. The patient table 17 of the magnetic resonance apparatus 10 moreover has a position determination apparatus 33, consisting of a distance sensor 30, a verification sensor 31 and a processing unit 32. The two sensors 30, 31 are connected to the processing unit 32. Measured data of the sensors 30, 31 can be forwarded to the processing unit 32 and stored by said unit and processed. At the same time the processing unit can define measuring tasks, in particular commands, and send them to the sensors 30, 31.

Due to the head-first position of the patient 15 there is no measurement of the head position by the sensors 30, 31 in FIG. 1, instead the head position of the patient is determined by the processing unit 32, for example via a contact acquisition, because of the position of the head coil 26. The position determination apparatus 33 can also comprise a sensor and/or tracker however, other than stated, which can detect or determine the position of the head coil. As an alternative the position of the head coil or of the patient's head can also be entered via the user interface 23 by a user. From the entered head position, the system control unit 22 or the processing unit 32 can calculate adherence to the limit values with regard to the local input of energy for or into the patient 15 as a result of the main magnetic field 14 generated by the main magnet 12, the gradient fields generated by the gradient coils 18 or the radio-frequency pulses generated by the radio-frequency antenna unit 20. Thus, in this exemplary embodiment, there is preferably no determination of the head position of the patient 15 by the distance sensor 30 or verification sensor 31.

The type of support of the patient 15 or the determination as to whether a head-first position (shown) or a feet-first position (not shown) is involved can be undertaken by the processing unit 33 or the system control unit 22. As an alternative, the type of support of the patient 15 can also be entered via the user interface 23. As an alternative, the type of support of the patient 15 can be determined by the verification sensor. For example, the verification sensor 31 can be embodied to recognize whether the nearest object involves the feet or the head of a patient 29.

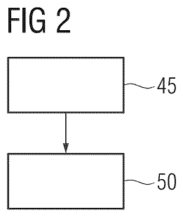

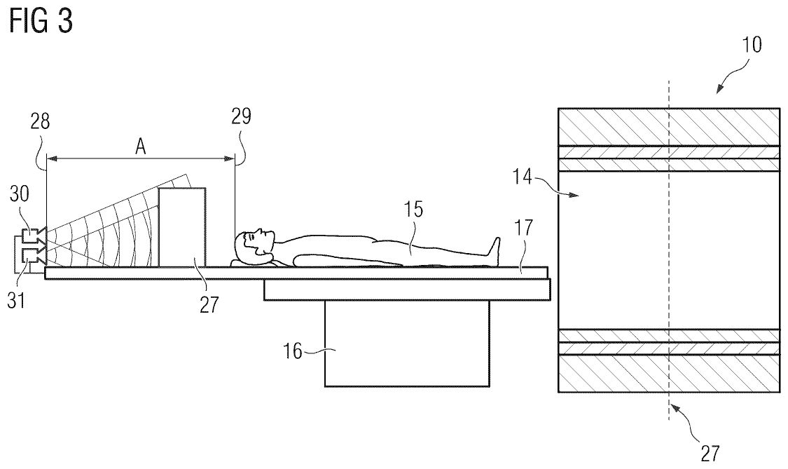

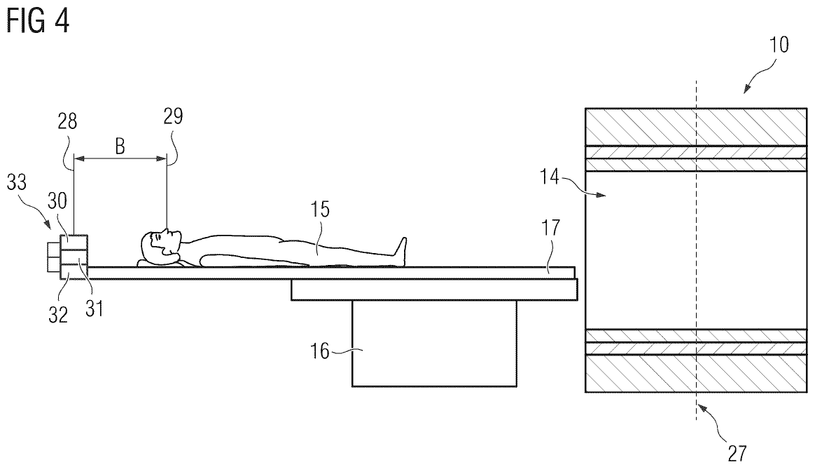

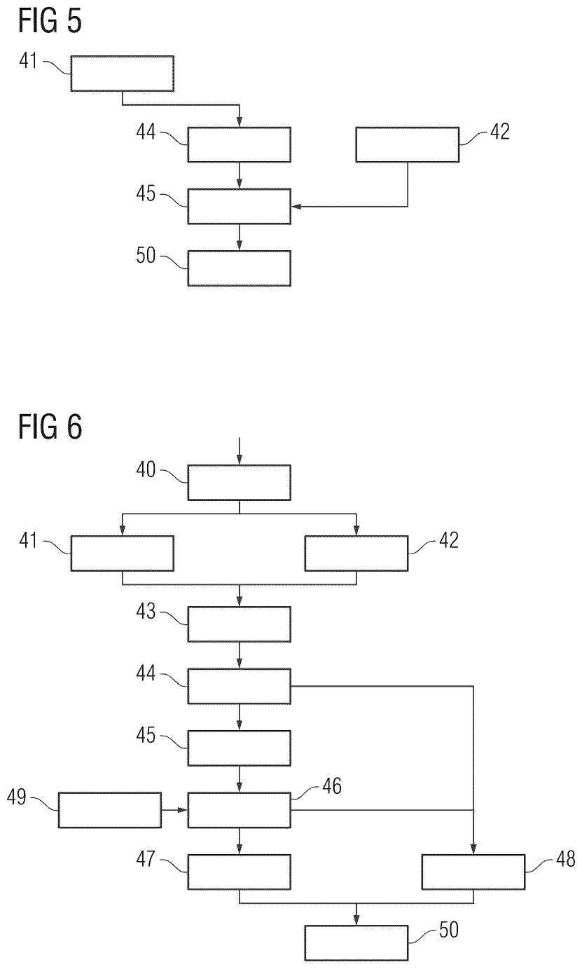

Shown in FIG. 2 is the process flow for checking a magnetic resonance sequence for adherence to at least one limit value. After the determination of the position information of a specific part of the body of a patient, in particular of the head, in step 45 the magnetic resonance sequence is checked for adherence of the at least one limit value with the aid of the position information of the specific part of the body, in particular of the head, in step 50. Further features of the method or method steps, in particular of the determination of the position information of the specific part of the body, are explained in FIGS. 5 and 6. The acquisition of distance measurement data and object measurement data by means of respective sensors is explained in FIGS. 3 and 4.

The patient 15 is located on the patient table 17 in FIG. 3 in a position referred to as a feet-first position. Usually with this type of support no head coil is used, but the patient 15 is positioned without any mechanical general conditions on the patient table. For example if the patient 15, in the diagram of FIG. 3, is rather closer to the head end of the patient table, or is supported at a greater distance A from reference point 28 (foot end of the patient table 17). The type of support of the patient 15 can be entered by an operator via a user interface 23 or recognized from a scan protocol or an examination instruction (automatically) by the magnetic resonance apparatus 10, or the system control unit 22. As an alternative a check or recognition of the type of support can be undertaken by the verification sensor 31.

The distance sensor 30 and the verification sensor 31 are attached to an end of the patient table 17 of the patient support apparatus 16. The sensors 30 and 31 are attached here to the end face side, referred to as the foot end, of the patient table 17. During the movement of the patient table 17 into the patient receiving area 14 of the magnetic resonance apparatus 10 within the framework of an examination (image recording), the sensors 30, 31 are thus always arranged spaced apart from the main magnet 12, the gradient coils 18 or the radio-frequency antenna unit 20. The sensors 30, 31 are thus not moved into the patient receiving area 14 (also examination area and/or magnetic field area) as well. In particular, the sensors 30, 31 are spaced apart from the isocenter 27 of the magnetic field area. The isocenter 27 in this case refers to the area that is located in the middle of the examination area (patient receiving area). Typically the magnetic fields are strongest in the isocenter 27. This means that in the isocenter 27 typically the energy which acts on the patient during an examination is at its greatest.

This form of embodiment of the magnetic resonance apparatus does not have a processing unit 32. The processing of the sensor signals or distance data and object data is undertaken by the system control unit 22, to which the sensors 30 and 31 are connected. The system control unit 22 can comprise a processing unit (embodied for this purpose).

The position of the head of the patient 29 can be determined by the vertex. The vertex here is the point on the head of the patient 29, to which the sensor arrangement 30, 31 is closest. Objects can (unintentionally) lie/be laid on the patient table 17 between the sensors 30, 31 and the head of the patient 29 17, such as for example in FIG. 3 the (obstacle) object 27. The objects can be of different sizes. In particular larger objects can adversely affect and/or prevent the distance and/or verification measurement. By its object properties, in particular geometry, the object 27 screens off the head of the patient 29, whereby the verification sensor 31 does not detect the head of the patient but instead the object 27. The distance measurement by means of the sensor 30 thus does not determine the head position of the patient 15 but instead that of the object 27. An (error) message is output and an estimated value or safety value is used for the examination.

In FIG. 4 too the patient 15 is located in a feet-first position on the patient table 17. The comparison between FIGS. 2 and 3 illustrates in particular the possible different positions of the patient 15 on the patient table 17. While in FIG. 2 a greater distance A between patient 15 and reference point 28 is depicted, in FIG. 3 a smaller distance B is shown. Moreover, in FIG. 4 there is no obstacle located between the patient 15 and the position determination apparatus 33.

The sensors, distance sensor 30 and verification sensor 31, and the processing unit 32 here form a joint module and/or component, the position determination apparatus 33. The position determination apparatus 33 is integrated into the patient support apparatus 16 or into the patient table 17. For example, the position determination apparatus 33 is built into a housing of the patient table 17. The reference point can therefore be determined as a point at the end of the patient table 17 at which the sensor apparatus or position determination apparatus 33 is arranged. Preferably here the midpoint of the component or module is selected as the reference point 28. The location (position) of the head of the patient 29 can likewise be described by the midpoint of the head.

Shown in FIG. 5 is a possible more detailed flow of the method steps of checking a magnetic resonance sequence for adherence to at least one limit value. On the basis of the object measurement data determined by a verification measurement 41 an object is recognized (verification of the head) 44. The verification measurement can, preferably, as illustrated in FIGS. 3 and 4, be undertaken by a verification sensor. A classification and/or object recognition can preferably be undertaken by a trained function. For example, a neural network can be trained in advance with corresponding object data marked by a medical expert. The neural network training can be continued during the examinations. To do this, object measurement data is preferably used without further patient information. The training and/or the results of the object recognition can be checked at regular intervals, for example by spot checks. Preferably there is a classification of the object into head of a patient or into another object. As an alternative there can also be further classifications, for example into object types.

The identification of the object 44 is followed by the determination of the distance 45 of the target object. By means of a distance sensor, in a distance measurement 42, preferably as illustrated in FIGS. 3 and 4, distance measurement data is determined. Preferably the distance measurement is already parallel with the object recognition 44 or the verification measurement 41. The distance measurement can however also not be undertaken until after the object recognition 44. The distance determination 45 is in particular undertaken on the basis of the distance measurement data, but can also be undertaken on the basis of the object measurement data or a combination of the two datasets.

Finally, in a method step 50 adherence to a limit value is checked. Here for example a maximum energy input due to the determined position of the head is calculated. This value is preferably compared with a threshold value and, if the threshold value is exceeded a warning is output. It is also possible to return to a positive result, i.e. the exceeding of the limit value.

Shown in FIG. 6 is the process flow of the checking of a magnetic resonance sequence for adherence to at least one limit value with additional intermediate and/or part steps. In a first step the location of a patient is detected 40. This can be undertaken by a user/operator of the magnetic resonance apparatus. As an alternative, the detection can also be undertaken by sensors, in particular distance sensors and/or verification sensors. Here a verification measurement 41 and a distance measurement 42 by means of respective sensors follow in parallel. After the merging of the measurement data 43 consisting of object measurement data and distance measurement data, the object detection 44 and the position determination 45 are undertaken. The merging of the measurement data here can be undertaken in a processing unit, in particular the processing unit of the position determination unit. For example, the object data can be compared with the distance measurement data and discrepancies can be stored. All information can be listed in a document, for example a text document, by means of a defined list (separated by spaces). The document containing the combined information from distance measurement and object measurement (verification measurement) can form the information base for the object detection 44 and the distance calculation (position determination) 55. If the head of a patient is detected or verified in the object detection 44 with adequate safety, calculation of the position 45 of the head of the patient is undertaken consecutively. In a checking step 46 the checking 46 of the position information can be undertaken. This can be undertaken manually by a user/operator or automatically by for example a neural network. The checking 46 can be undertaken on the basis of patient information 49 provided. Other previous examinations, the type of examination, the user or environmental conditions can also be taken into consideration, however. If the verification of the head cannot be undertaken, or if an object other than the head of the patient 15 is detected, the return to an estimated value 48 is carried out from verification step 44. If in the checking of the position information 46 irregularities, errors or uncertainties are detected, the return to an estimated value 48 is likewise carried out. With a positive verification 44 and check 46 the position value of the head of the patient is output. Finally there is the check as to whether a limit value for the energy absorption rate for the head of the patient 15 has been exceeded.

It is pointed out once again in conclusion that the method described in detail above, as well as the magnetic resonance apparatus shown, merely involve exemplary embodiments, which can be modified by the person skilled in the art in a very wide variety of ways without departing from the field of the invention. Furthermore the use of the indefinite article “a” or “an” does not exclude the features concerned also being able to be present a number of times. Likewise the terms “unit” and “element” do not exclude the components concerned consisting of a number of interacting part components, which where necessary can also be spatially distributed. Independent of the grammatical term usage of a specific person-related term, individuals with male, female or other gender identities should be included within the term.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for checking a magnetic resonance sequence for adherence to at least one limit value, the method comprising:
determining position information of a specific part of a body of a patient on a patient table; and
checking the magnetic resonance sequence for adherence to the at least one limit value using the position information of the specific part of the body.

2. The method of claim 1, further comprising:
acquiring distance measurement data for measurement of a distance between a target object and a reference object of the patient table, wherein the determining determines the position information of the specific part of the body of the patient using the distance measurement data.

3. The method of claim 2, wherein the acquiring acquires the distance measurement data using a distance sensor.

4. The method of claim 3, wherein the distance sensor comprises an ultrasound sensor.

5. The method of claim 3, wherein the distance measurement data comprises at least one of two-dimensional distance measurement data or three-dimensional distance measurement data.

6. The method of claim 2, further comprising:
acquiring object measurement data; and
verifying the target object as a head of the patient using the object measurement data.

7. The method of claim 6, wherein the acquiring the object measurement data acquires the object measurement data using a verification sensor.

8. The method of claim 7, wherein the verification sensor comprises a temperature sensor.

9. The method of claim 7, wherein the object measurement data comprises at least one of two-dimensional object measurement data or three-dimensional object measurement data.

10. The method of claim 6, wherein the determining the position information of the specific part of the body of the patient is based on the object measurement data.

11. The method of claim 1, further comprising:
providing patient data, wherein the determining the position information of the specific part of the body of the patient is based on the patient data.

12. The method of claim 1, wherein a defined estimated value is defined as position information if the position information of the specific part of the body of the patient cannot be determined with at least one of adequate safety or accuracy.

13. The method of claim 1, wherein the specific part is a head of the patient and the determining the position information of the head comprises an object classification method, the object classification method including a computer-implemented, trained function, including,
inputting object measurement data into the trained function,
applying the trained function, the trained function configured to detect and to classify objects using the object measurement data, and
returning a classification of an object detected using the trained function.

14. A magnetic resonance apparatus comprising:
a magnet unit surrounding a patient receiving area;
a patient table movable into the patient receiving area; and
a position determination apparatus configured to perform the method of claim 1.

15. The magnetic resonance apparatus of claim 14, wherein the position determination apparatus is on the patient table at a foot end of the patient table.

16. The method of claim 1, wherein the position information of the specific part of the body of the patient includes position information of a head of the patient.

17. The method of claim 2, wherein the target object is a head of the patient and the reference object is a reference point of the patient table.

18. The method of claim 7, wherein the determining the position information of the specific part of the body of the patient is based on the object measurement data.

19. The method of claim 18, further comprising:
providing patient data, wherein the determining the position information of the specific part of the body of the patient is based on the patient data.

20. The method of claim 19, wherein a defined estimated value is defined as position information if the position information of the specific part of the body of the patient cannot be determined with at least one of adequate safety or accuracy.
